(19) United States
(12) Patent Application Publication
(10) Pub. No.: US 2026/0198443 A1
(43) Pub. Date: Jul. 16, 2026

(54) INBRED RICE LINE DG563PVL

(71) Applicant: Nutrien AG Solutions, Inc., Loveland, CO (US)

(72) Inventors: Randy Ouzts, Loveland, CO (US); Qiming Shao, Sugar Land, TX (US); Nanyen Chou, Sugar Land, TX (US)

(73) Assignee: Nutrien AG Solutions, Inc., Loveland, CO (US)

(21) Appl. No.: 19/022,452

(22) Filed: Jan. 15, 2025

(51) Int. Cl. A01H 6/46; A01H 5/10; A01N 43/60; A01P 13/00

(52) U.S. Cl. CPC A01H 6/4636; A01H 5/10; A01N 43/60; A01P 13/00

(57) ABSTRACT

A novel inbred rice line designated DG563PVL and seed, plants and plant parts thereof are provided. Methods for producing a rice plant comprise crossing rice variety DG563PVL with another rice plant are provided. Method for producing a rice plant containing in its genetic material one or more traits introgressed into DG563PVL through backcross conversion and/or transformation, and to the rice seed, plant and plant part produced thereby are provided.

### BACKGROUND

An ancient agricultural crop, rice remains one of the world's principal food crops.

Rice in the United States is classified into three primary market types by grain size, shape, and endosperm composition: long-grain, medium-grain, and short-grain. Typical U.S. long-grain cultivars cook dry and fluffy when steamed or boiled, while medium- and short-grain cultivars cook moist and sticky. Medium grain rice is usually slightly wider than long grain rice and slightly shorter, with a length:width ratio between 2:1 and 3:1.

Rice is a semi-aquatic crop that benefits from flooded soil conditions during part or all of the growing season. In the rice producing regions of the United States, rice is typically grown on flooded soil to optimize grain yields.

Long-grain cultivars have become the principal varieties grown throughout the southern United States.

A goal of rice breeders is to develop stable, high yielding rice cultivars that are agronomically sound. The reasons for this goal are to maximize the amount of grain produced on the land used and to supply food for both animals and humans. To accomplish this goal, the rice breeder must select and develop rice plants that have the traits that result in superior cultivars.

### SUMMARY

Rice plants and seeds of line DG563PVL are provided, as are plant parts, tissue cultures of cells, breeding methods, and hybrid rice seed and plants. Inbred rice line designated DG563PVL is a semi-dwarf, early maturing long grain rice variety (Oryza sativa L.).

Plants and plant parts having the genotypic and phenotypic characteristics of inbred line DG563PVL is described herein.

Rice plants and seeds of line DG563PVL having resistance and/or tolerance to at least one ACCase-inhibiting aryloxyphoxypropanoate herbicide composition.

Method of breeding with DG563PVL, including selection for progeny having resistance and/or tolerance to at least one ACCase-inhibiting aryloxyphoxypropanoate herbicide composition.

Method for growing a plant having the genotypic and phenotypic characteristics of DG563PVL, including applying at least one ACCase-inhibiting aryloxyphoxypropanoate herbicide composition during the growing season.

Method for controlling weeds in the vicinity of Rice plants and seeds of line DG563PVL, including applying at least one ACCase-inhibiting aryloxyphoxypropanoate herbicide composition in the vicinity.

Industrial uses for DG563PVL and commodity products derived from plants and plant parts of DG563PVL are provided.

### DETAILED DESCRIPTION

The present disclosure provides a new inbred rice line designated DG563PVL. Rice plants and seeds of line DG563PVL are provided, as are plant parts, tissue cultures of cells, breeding methods, and hybrid rice seed and plants.

The seed of inbred rice line DG563PVL, the plant produced from the inbred rice line DG563PVL seed, the hybrid rice plant produced from the crossing of the inbred rice line DG563PVL, and various parts of the inbred rice line DG563PVL and hybrid rice plants and transgenic versions of the foregoing can be utilized for human food, livestock feed, and as a raw material in industry. DG563PVL and its hybrids and derived varieties are adapted for growing throughout the rice-growing areas of Texas, Louisiana, Arkansas, Mississippi, Missouri, and Florida, and will also be well suited for growing in many other rice-producing areas throughout the world.

### Definitions

As used herein, the following terms are intended to have the following meanings:

ACCase-inhibiting aryloxyphoxypropanoate herbicide composition: ACCase-inhibiting aryloxyphyoxypropanoate herbicide composition is one of three classes of herbicide composition classes that can inhibit Acetyl-Coenzyme A carboxylase (ACCase) in grasses. Examples of ACCase-inhibiting aryloxyphoxypropanoate herbicinde compositions include, but are not limited to, quizalofop or an ester thereof, haloxyfop, fluazifop or an ester thereof, clodinafop, clodinafop-propargyl, diclofop, and diclofop-methyl.

Amylose: Amylose is a type of grain starch that affects cooking behavior. As such, its measured quantity in rice is used to establish cooking properties of Standard US grain classes, or types (long, medium and short grain).

Amylose Percent: “Amylose percent” refers to the percentage of the endosperm starch of milled rice that is amylose. Standard long grains contain 20 to 23 percent amylose. Rexmont-type long grains contain 24 to 25 percent amylose. Short and medium grains contain 14 to 16 percent amylose. Waxy rice contains zero percent amylose. Amylose values, like most characteristics of rice, will vary over environments.

Cell: The term “cell” generally refers to a plant cell, whether isolated, in tissue culture, or included in a plant or plant part.

Chalk: “Chalk” refers to an opaque region of the rice kernel resulting from loose packing of the starch granules. Chalk may occur throughout or in a part of the kernel.

Days to 50% Heading: The term “days to 50% heading” refers to the number of days from emergence to the day when 50% of all panicles are exerted at least partially through the leaf sheath. “Days to 50% heading” is a measure of growth duration.

Gelatinization temperature: “Gelatinization temperature” refers to the temperature at which the consistency of a rice flour-water mixture changes into a jelly. Correlates with the cooking time and texture of a rice product.

Genetically modified: “Genetically modified” refers to an organism that has received genetic material from another, or had its genetic material modified, resulting in a change in one or more of its phenotypic characteristics. Methods used to modify, introduce or delete the genetic material may include mutation breeding, genome editing, backcross conversion, genetic transformation, introgression, single and multiple gene conversion, and/or direct gene transfer.

Introgression: The process of transferring genetic material from one genotype to another.

Plant: As used herein, the term “plant” includes reference to immature or mature whole plant, including a plant from which seed or grain has been removed. Seed or embryo that will produce the plant is also considered to be the plant.

Plant part: As used herein, the term “plant part” includes plant cells, plant protoplasts, plant cell tissue cultures from which rice plants can be regenerated, plant calli, plant clumps and plant cells that are intact in plants, such as embryos, pollen, ovules, flowers, glumes, panicles, leaves, stems, roots, root tips, anthers, pistils and the like. In some embodiments, the plant part contains at least one cell of inbred rice line DG563PVL. Another aspect of the present disclosure is to provide for cells which upon growth and differentiation produce a cultivar having essentially all of the physiological and morphological characteristics of inbred rice line DG563PVL. Genetic variants of inbred rice line DG563PVL can also be obtained as a result of the tissue culture process. Variants recovered by tissue culture of inbred rice line DG563PVL are another aspect of this invention.

Tolerant: “Tolerant” or “herbicide tolerant” indicates a plant or portion thereof capable of growing in the presence of an amount of herbicide that normally causes growth inhibition in a non-tolerant (e.g., a wild-type) plant or portion thereof. Levels of herbicide that normally inhibit growth of a non-tolerant plant are known and readily determined by those of skill in the art. Examples include the amounts recommended by manufacturers for application. The maximum rate is an example of an amount of herbicide that would normally inhibit growth of a non-tolerant plant. For the present application, the terms “tolerant” and “resistant” are used interchangeably and are intended to have an equivalent meaning and an equivalent scope. Similarly, the terms herbicide-tolerant and herbicide-resistant are used interchangeably and are intended to have equivalent meaning and an equivalent scope. The terms “herbicide-tolerance” and “herbicide-resistance” are also used interchangeably and are intended to have equivalent meaning and equivalent scope.

Weed Killing Rate: “Weed killing rate”, “effective rate,” and “effective amount” are used interchangeably and are intended to have equivalent meaning and equivalent scope. The effective amount is the amount of an active ingredient that will inhibit the growth of a non-tolerant plant, such as a weed, but not negatively impact or inhibit the growth of a tolerant plant.

### Variety Description

Inbred rice line designated DG563PVL is a semi-dwarf, early maturing long grain rice variety (Oryza sativa L.). DG563PVL has a broad germplasm background with excellent yield potential, and stable yields across trials. The line has 22.9% amylose, with an intermediate gel temperature, low chalk content, and demonstrated good yield and excellent milling attributes in multi-state testing in southern United States rice production areas. DG563PVL typically shows 84-92 days from planting to 50% heading or about 87 average days from planting to 50% heading. DG563PVL is 97 centimeters in height at maturity.

DG563PVL is tolerant of herbicides that inhibit acetyl-Coenzyme A carboxylase activity, such as the herbicide quizalofop and esters thereof. This herbicide tolerant phenotype exhibited by the inbred rice variety DG563PVL is due to a mutant ACCase gene bred into DG563PVL through introgression of the gene from parent line PVL01. U.S. Pat. No. 12,048,281, incorporated by reference herein, describes a mutant ACCase gene present in PVL01. Despite the introgression of the mutant ACCase gene, DG563 exhibits no fitness penalty as compared to its DG263L parent.

Inbred rice line DG563 PVL was observed to possess the physiological and morphological characteristics set out in Table 1, based primarily on averages of tests conducted over 2024 at multiple sites (El Campo, Texas; Bay City, Texas; Winterville, Mississippi; Bernie, Missouri, McNess, Louisiana; Lake Arthur, Louisiana; Mamou, Louisiana; Crowley, Louisiana; South Farm, Louisiana; and Palmetto, Louisiana).

TABLE 1
Physiological and Morphological Characteristics of Inbred Rice Line
DG563PVL
Type
Rice Type: Long grain
Endosperm
Endosperm Type: Non-Glutinous
Endosperm Content of Amylose: Medium
Coleoptile
Coleoptile Anthocyanin Coloration: Medium
Plant Growth Habit: Erect
Leaf Sheath
Leaf Sheath Distal Leaf Sheath Anthocyanin Coloration: Medium to Strong
Leaf Sheath Basal Sheath Anthocyanin Coloration: Medium
Leaf Blade
Leaf Blade Intensity of Green Color: Very Light
Leaf Blade Anthocyanin Coloration: Absent
Leaf Blade Pubescence: Absent or Very Sparse
Ligule
Ligule Color: White
Ligule Shape: Cleft
Timing
Timing of Panicle Emergence: Early to Medium
Time of Maturity (Days after Planting): 124
Time of Senescence (Days after Planting): 135
Flag Leaf
Flag Leaf Length of Blade: Medium
Flag Leaf Length of Blade (cm): 32
Flag Leaf Width of Blade: Medium
Flag Leaf Width of Blad (mm): 15
Lemma
Lemma Pubescence: Sparce
Lemma Color: Yellowish
Lemma Coloration with Phenol: Absent or Very Weak
Stigma
Stigma color: White
Stem
Stem Length: Medium
Stem Length (cm): 75
Stem Thickness: Medium
Stem Thickness (cm): 0.7
Stem Intensity of anthocyanin coloration of Nodes: Absent or Very Weak
Stem Anthocyanin Coloration of Internodes: Absent or Very Weak
Panicle
Panicle Number of Panicles: Medium to Many
Panicle Number of Panicles: 9
Panicle Distribution of Awns: Absent
Panicle Awns Length: Very Short
Panicle Awns Length (mm): 0
Panicle Length: Medium to Long
Panicle Length (cm): 24
Panicle Density: Medium to Dense
Panicle Attitude: Semi-Drooping
Panicle Attitude of Branches: Erect to Semi-Erect
Panicle Number of Secondary Branches: Medium
Panicle Number of Secondary Branches (#): 5
Panicle Exsertion: Well Exserted
Timing
Time of Maturity: Early to Medium
Time of Senescence: Medium
Time of Panicle Emergence (Days after Planting): 87
Glume
Glume Length: Medium
Glume Color: Yellowish
Culm
Culm Angle (degrees from perpendicular after flowering): Erect (less than 30°)
Culm Length or Plant Height (cm): 95
Grain Spikelet
Awns after Full Heading: Absent
Apiculus Color at Maturity: White
Grain Seed
Seed Weight per 1000 Seeds: Medium to High
Seed Weight per 1,000 Seeds (g): 24
Grain Length: Long
Grain Length (mm): 8.5
Grain Width: Medium
Grain Width: 2.7
Grain Ratio Length/Width: Medium to High
Grain Ratio Length/Width: 3.14
Grain Color: Light Brown
Grain Alkali Digestion: Absent or Very Weak
Grain Aroma: Absent or Weak
Endosperm Type: Non-glutinous (non-waxy)
Milling Quality (% hulls): 18.8
Milling Yield (% white kernel (head) rice to rough rice): 62
% Protein: 7.2
% Amylose: 22.9
Alkali Spreading Value: 4.1% KOH to 1.7% KOH
Gelatination Temperature Type: Intermediate
Grain Amylographic Paste Viscosity Peak: 68
Resistance to Low Temperature
Low Temperature Germination Vigor and Seedling Vigor: Medium
Low Temperature Flowering Spikelet Fertility: Medium
Seedling Vigor
Seedling Vigor: Medium
Disease Resistance
Narrow Brown Leaf Spot (Cerospora oryzae): Moderately Resistant
Sheath Blight (Rhizoctonia solani): Moderately Susceptible
Insect Resistance
Rice Stink Bug (Oegalus pugnax): Intermediate
Rice Water Weevil (Lissorhoptrus oryzophilus): Moderately Susceptible

Comparisons of Rice Line DG563PVL with Other Rice Lines

Tables 2 to 6 present comparisons of agronomic characteristics and quality ratings of inbred rice line DG563PVL to rice lines PVL03 and DG263L. The variety PVL03 was provided by Louisiana State University. DG263L is a rice variety of Loveland Products, Inc.

In Table 2, the average days to 50% heading (days) for DG563PVL and PVL03 at Multi-location yield trials in Texas, Louisiana, Arkansas, and Mississippi in 2024.

TABLE 2
Average Days to 50% Heading (Days) at Multi-Location Yield Trials

| Year | Location | DG563PVL | PVL03 |
|---|---|---|---|
| 2024 Nutrien | NAS-El Campo, TX early | 70 | 64 |
| Ag Solution Test | NAS-El Campo, TX late | 84 | 78 |
| | Winterville, MS | 89 | 87 |
| | Zack Farm, Bernie, MO | 95 | 98 |
| | UA-Stuttgart, AR | 81 | 80 |
| | Research Farm, Campell, MO | 86 | 87 |
| Grand Mean | | 84 | 82 |

In Table 3, the average days to 50% heading (days) for DG563PVL, PVL03 and DG263L in Pre-Commercial trials in El Campo, Texas; Bay City, Texas; Winterville, Mississippi; and Bernie, Missouri in 2024.

TABLE 3
Average Days to 50% Heading (Days) in Pre-Commercial
Trials in Texas, Mississippi, and Missouri

| Year | Locations | DG563PVL | PVL03 | DG263L |
|---|---|---|---|---|
| 2024 NAS Tests | NAS-El Campo, TX | 96 | 85 | 90 |
| | NAS-Bay City, TX | 88 | 86 | 86 |
| | Winterville, MS | 89 | 88 | 88 |
| | Zack Farm, Bernie, MO | 94 | 95 | 92 |
| Grand Mean | | 92 | 89 | 89 |

In Table 4, the average plant height (cm) for DG563PVL, PVL03 and DG263L in Pre-Commercial Trials in El Campo, Texas; Bay City, Texas; Winterville, Mississippi; Bernie, Missouri, McNess, Louisiana; Lake Arthur, Louisiana; Mamou, Louisiana; Crowley, Louisiana; South Farm, Louisiana; and Palmetto, Louisiana in 2024.

TABLE 4
Average Plant Height (cm) in Pre-Commercial Trials in
Texas, Mississippi, Missouri, and Louisiana

| | Locations | DG563PVL | PVL03 | DG263L |
|---|---|---|---|---|
| Nutrien Ag | NAS-El Campo, TX | 87 | 96 | 81 |
| Solutions | NAS-Bay City, TX | 90 | 96 | 91 |
| | Winterville, MS | 108 | 108 | 106 |
| | Zack Farm, Bernie, MO | 95 | 93 | 97 |
| Louisiana State | LSU-McNess, LA | 96 | 109 | 96 |
| University | LSU-Lake Arthur, LA | 96 | 101 | 91 |
| | LSU-Mamou, LA | 98 | 103 | 105 |
| | LSU-Crowley, LA Early | 103 | 114 | 103 |
| | LSU-Crowley, LA Late | 95 | 105 | 102 |
| | LSU-South farm, LA | 99 | 101 | 99 |
| | LSU-Palmetto, LA | 101 | 103 | 87 |
| Grand Mean | | 97 | 103 | 96 |

In Table 5, the average whole grain milling data for DG563PVL, PVL03, and DG263L in Pre-Commercial Trials in 2024 in El Campo, Texas; Bay City, Texas; Winterville, Mississippi; Bernie, Missouri, McNess, Louisiana; Lake Arthur, Louisiana; Mamou, Louisiana; Crowley, Louisiana; South Farm, Louisiana; and Palmetto, Louisiana.

TABLE 5
Average Whole Grain Milling Data in Pre-Commercial Trials
in Texas, Mississippi, Missouri, and Louisiana

| | Locations | DG563PVL | PVL03 | DG263L |
|---|---|---|---|---|
| Nutrien Ag | NAS-El Campo, TX | 62 | 62 | 60 |
| Solutions | NAS-Bay City, TX | 59 | 59 | 59 |
| | Winterville, MS | 59 | 58 | 59 |
| | Zack Farm, Bernie, MO | 58 | 49 | 63 |
| Louisiana State | LSU-McNess, LA | 54 | 52 | 50 |
| University | LSU-Lake Arthur, LA | 56 | 53 | 55 |
| | LSU-Mamou, LA | 59 | 52 | 54 |
| | LSU-Crowley, LA Early | 61 | 61 | 58 |
| | LSU-Crowley, LA Late | 59 | 53 | 58 |
| | LSU-South farm, LA | 56 | 50 | 58 |
| | LSU-Palmetto, LA | 59 | 55 | 59 |
| Grand Mean | | 58 | 55 | 58 |

In Table 6, the average total milling grain milling data for DG563PVL, PVL03, and DG263 in Pre-Commercial Trials in 2024 in El Campo, Texas; Bay City, Texas; Winterville, Mississippi; Bernie, Missouri, McNess, Louisiana; Lake Arthur, Louisiana; Mamou, Louisiana; Crowley, Louisiana; South Farm, Louisiana; and Palmetto, Louisiana.

TABLE 6
Average Total Milling Grain Data

| | Locations | DG563PVL | PVL03 | DG263L |
|---|---|---|---|---|
| Nutrien Ag | NAS-El Campo, TX | 68 | 65 | 65 |
| Solutions | NAS-Bay City, TX | 67 | 69 | 66 |
| | Winterville, MS | 67 | 69 | 67 |
| | Zack Farm, Bernie, MO | 67 | 69 | 68 |
| Louisiana State | LSU-McNess, LA | 68 | 68 | 65 |
| University | LSU-Lake Arthur, LA | 67 | 69 | 66 |
| | LSU-Mamou, LA | 71 | 71 | 69 |
| | LSU-Crowley, LA Early | 68 | 71 | 67 |
| | LSU-Crowley, LA Late | 68 | 69 | 68 |
| | LSU-South farm, LA | 67 | 66 | 67 |
| | LSU-Palmetto, LA | 70 | 72 | 69 |
| Grand Mean | | 68 | 69 | 67 |

### Breeding History of DG563PVL

DG536PVL was developed from F₀ crosses made using PVL01×DG263L. BC₁F₁ was made using DG263L as recurrent parent (PVL01/DG263L/2/DG263L), plus the molecular marker assistant help. Following the cross, plants were selected for characteristics similar to DG263L, such as strong straw, relatively short height, big panicles, good tillers, high yield potential, good milling and grain quality, and good pollinator ability. BC₂F₁ was made using DG263L as recurrent parent (PVL01/DG263L/2/DG263L/3/DG263L), plus the molecular marker assistant help. Additionally, 18 oz/ac Provisia herbicide, having the active ingredient quizalofop-p-ethyl, was applied in the BC₂F₁ populations to for herbicide tolerance due to the presence of a mutant ACCase gene, referred to herein as PV, present in the PVL01 parent variety. BC2F2 populations were planted in the screenhouse, early panicles were taken and planted. From these selections, DG563PVL was identified. It was entered into the yield trials and screening with Provisia herbicide. DG563PVL has shown uniformity and stability. The line has been self-pollinated a sufficient number of generations with careful attention to uniformity of plant type and has been increased with continued observation for uniformity.

Breeding with Inbred Rice Line DG563PVL

In some embodiments, rice varieties such as DG563PVL provide a source of breeding material that may be used to develop new rice inbred varieties. Plant breeding techniques known in the art and used in a rice plant breeding program include, but are not limited to, recurrent selection, mass selection, bulk selection, backcrossing, pedigree breeding, open pollination breeding, restriction fragment length polymorphism (RFLP) enhanced selection, genetic marker enhanced selection, making double haploids and transformation. Often combinations of these techniques are used. There are many analytical methods available to evaluate the result of a cross. The oldest and most traditional method of analysis is the observation of phenotypic traits, but genotypic analysis may also be used.

The present disclosure provides methods for producing a rice seed by crossing a first parent rice plant with a second parent rice plant, where the first or the second parent rice plant is a rice plant of inbred rice line DG563PVL, with the other parent rice plant being of a different rice line. Also provided are methods for producing a rice seed where both the first and the second parent rice plants are rice plants of inbred rice line DG563PVL. The present disclosure thus provides a multitude of breeding methods involving DG563PVL, including, for example, selfing, backcrosses, hybrid production, crosses to populations, and the like.

The present disclosure also provides methods for producing a rice plant by crossing a rice plant of line DG563PVL with a second, different rice plant and growing the resultant progeny seed, and repeating the crossing and growing steps with DG563PVL progeny plants from 0 to 7 times. All plants produced using inbred rice line DG563PVL are within the scope of the present disclosure, including those developed from varieties derived from inbred rice line DG563PVL.

Inbred rice line DG563PVL can be used in the development of further rice plants. In one embodiment, a method for developing a DG563PVL-derived progeny rice plant in a rice breeding program includes utilizing a plant of line DG563PVL or a plant part thereof as a source of breeding material and selecting a progeny plant with morphological and/or physiological characteristics selected from those set out in Tables 1 to 6, and/or selecting a progeny plant with molecular markers in common with inbred rice line DG563PVL. Breeding methods or steps that can be used in a rice breeding program involving DG563PVL include pedigree breeding, backcrossing, mutation breeding, and recurrent selection. In addition to these methods, techniques such as RFLP-enhanced selection, genetic marker-enhanced selection (e.g., SSR markers), and the making of double haploids can be used.

Other methods include crossing a plant of inbred line DG563PVL with a rice plant of a different line, thereby producing a population of rice plants, which, on average, derive 50% of their alleles from rice line DG563PVL. A plant of the resulting population of rice plants can be selected and repeatedly selfed or sibbed with a rice cultivar resulting from these successive filial generations. The present disclosure thus provides a rice cultivar produced by such a method, where the resulting rice cultivar has obtained at least 50% of its alleles from DG563PVL.

A person of ordinary skill in the art of rice breeding would know how to evaluate the traits of two rice varieties to determine whether there is any significant difference between the traits express by the two rice varieties. See, for example, Fehr and Walt (1987), Principles of Cultivar Development, pp. 261-286. The present disclosure thus provides DG563PVL progeny plants comprising a combination of at least two DG563PVL traits selected from those provided in any of Tables 1 to 6, so that the DG563PVL progeny rice plant is not significantly different for the selected trait that inbred rice line DG563PVL. Molecular markers can be used to identify a progeny plant as a DG563PVL progeny plant. Mean trait values can be used to determine whether trait differences are significant. In some embodiments, the traits are measured on plants grown under the same environmental conditions.

Embodiments of the present disclosure also include selection of progeny of a rice seed derived from the variety DG563PVL by applying a weed-killing rate of ACCase-inhibiting herbicide during the growing of the progeny seed to identify progeny displaying the herbicide tolerance of DG563PVL.

Progeny of DG563PVL can also be characterized through their filial relationship with inbred rice line DG563PVL. A breeding cross is a cross made to introduce new genetics into the progeny plants and is distinguished from a cross such as a self or a sib cross made to select among existing genetic alleles. The lower the number of breeding crosses in the pedigree, the closer the relationship between inbred rice line DG563PVL and its progeny. Progeny plants produced by the methods described herein can be within 1, 2, 3, 4, or 5 breeding crosses of inbred rice line DG563PVL.

### Pedigree Breeding

Pedigree breeding starts with the crossing of two genotypes, such as inbred rice line DG563PVL and a rice plant of another line or variety having one or more desirable characteristics that is lacking in, or which complements, inbred rice line DG563PVL. If the two original parents do not provide all the desired characteristics, other sources can be included in the breeding population. In the pedigree method, superior plants are selfed and selected in successive filial generations. In the succeeding filial generations, the heterozygous condition gives way to homogeneous varieties as a result of self-pollination and selection. Typically, in the pedigree method of breeding, five or more successive filial generations of selfing and selection is practiced: F1 to F2; F2 to F3; F3 to F4; F4 to F5, etc. After sufficient inbreeding, successive filial generations will serve to increase seed of the developed variety. Preferably, the developed variety comprises homozygous alleles at about 95% or more of its loci.

In addition to being used to create a backcross conversion, backcrossing can also be used in combination with pedigree breeding. Backcrossing can be used to transfer one or more specifically desirable traits from one variety, the donor parent, to a developed variety called the recurrent parent, which has overall good agronomic characteristics yet lacks that desirable trait or traits. However, the same procedure can be used to move the progeny toward the genotype of the recurrent parent but at the same time retain many components of the non-recurrent parent by stopping the backcrossing at an early stage and proceeding with selfing and selection. For example, a rice variety may be crossed with another rice variety to produce a first generation progeny plant. The first generation progeny plant may then be backcrossed to one of its parent varieties to create a BC1 or BC2. Progeny are selfed and selected so that the newly developed variety has many of the attributes of the recurrent parent and yet several of the desired attributes of the non-recurrent parent. This approach leverages the value and strengths of the recurrent parent for use in new rice varieties.

Methods of making a backcross conversion of inbred rice line DG563PVL are provided. Such methods include the steps of crossing a plant of inbred rice line DG563PVL with a donor plant comprising a desired trait, selecting an F1 progeny plant comprising the desired trait, and backcrossing the selected F1 progeny plant to a plant of inbred rice line DG563PVL. Such methods can include the step of obtaining a molecular marker profile of inbred rice line DG563PVL and using the molecular marker profile to select a progeny plant with the desired trait and the molecular marker profile of inbred rice line DG563PVL. In one embodiment, the desired trait is a mutant gene or transgene present in the donor parent.

### Hybrid Breeding

The inbred rice line DG563PVL can be used in the production of a first-generation (i.e., F1) hybrid plant. Methods for producing such an F1 hybrid include crossing a first parent rice plant with a second parent rice plant, wherein either the first or the second parent rice plant is a DG563PVL plant and the other parent rice plant is a different rice plant. Also provided are methods for producing a hybrid rice line derived from DG563PVL by crossing DG563PVL with a different rice plant and growing the progeny seed. The crossing and growing steps can be repeated any number of times.

Optionally, either the first or the second parent rice plant can be produced in male-sterile form, using techniques known in the art.

DG563PVL can be used in both two-line hybrid rice breeding and three-line hybrid rice breeding. Both methods are well known in the art.

### Recurrent Selection and Mass Selection

Recurrent selection is a method used in a plant breeding program to improve a population of plants. Inbred rice line DG563PVL is suitable for use in a recurrent selection program. Recurrent selection involves individual plants cross pollinating with each other to form progeny. The progenies are grown and the superior progeny selected by any number of selection methods, which include individual plant, half-sib progeny, full-sib progeny and selfed progeny. The selected progeny are cross pollinated with each other to form progeny for another population. The resulting population is planted and superior plants are again selected to cross pollinate with each other. Recurrent selection is a cyclical process and therefore can be repeated as many times as desired. The objective of recurrent selection is to improve the traits of a population. The improved population can then be used as a source of breeding material to obtain new varieties for commercial or breeding use, including the production of a synthetic cultivar. A synthetic cultivar is the resultant progeny formed by the inter-crossing of several selected varieties.

Mass selection is a useful technique when used in conjunction with molecular marker enhanced selection. In mass selection, seeds from individuals are selected based on phenotype or genotype. These selected seeds are then bulked and used to grow the next generation. Bulk selection requires growing a population of plants in a bulk plot, allowing the plants to self-pollinate, harvesting the seed in bulk and then using a sample of the seed harvested in bulk to plant the next generation. Instead of self-pollination, directed pollination could be used as part of the breeding program.

### Mutation Breeding

Mutation breeding can be used to introduce new traits into inbred rice line DG563PVL. Mutations that occur spontaneously or are artificially induced can be useful sources of variability for a plant breeder. The goal of artificial mutagenesis is to increase the rate of mutation for a desired characteristic. Mutation rates can be increased by many different means including, for example, temperature; long-term seed storage; tissue culture conditions; radiation, such as X-rays, Gamma rays (e.g. cobalt 60 or cesium 137), neutrons, (product of nuclear fission by uranium 235 in an atomic reactor), Beta radiation (emitted from radioisotopes such as phosphorus 32 or carbon 14), or ultraviolet radiation (preferably from 2500 to 2900 nm); or chemical mutagens such as base analogues, (5-bromo-uracil), related compounds (8-ethoxy caffeine), antibiotics (streptonigrin), alkylating agents (sulfur mustards, nitrogen mustards, epoxides, ethylenamines, sulfates, sulfonates, sulfones, lactones), azide, hydroxylamine, nitrous acid, or acridines. Once a desired trait is observed through mutagenesis, the trait may then be incorporated into existing germplasm by traditional breeding techniques. Mutations created in other rice plants may be used to produce a backcross conversion of inbred rice line DG563PVL that includes the mutation.

### Marker Assisted Breeding

Isozyme Electrophoresis and RFLPs have been widely used to determine genetic composition. See for example, Dinka et al. (2007), Genetics, 176 (4): 2035-54; Gonzalez et al. (2007) Mol. Plant Microbe Interact. 20 (5): 534-546; Jin et al. (2006), Plant Mol. Biol. 62 (4-5): 769-777; Pan et al. (2006), Plant Mol. Biol. 61 (6): 933-943; and Huang et al. (2007), J. Genet. Genomics, 33 (4): 330-338.

SSR technology is one of the most efficient and practical marker technologies. More marker loci can be routinely used and more alleles per marker locus can be found using SSRs in comparison to RFLPs. See, e.g., Lawson et al. (2006), Genome Biology, 7: R14; Nagaraju et al. (2002), PNAS USA, 99 (9): 5836-5841; and Lu et al. (2005), Crop Science. 45:66-76. Single nucleotide polymorphisms (SNPs) may also be used to identify the unique genetic composition of DG563PVL plants and progeny varieties retaining the unique genetic composition of DG563PVL. Various molecular marker techniques may be used in combination to enhance overall resolution.

Rice DNA molecular marker linkage maps have been rapidly constructed and widely implemented in genetic studies such as in Zhu et al. (1999), Mol. Gene Genetics, 261 (1): 184-195; Cheng et al. (2001), Genome Research, 11 (12): 2133-2141; Ahn et al. (1993), PNAS USA, 90 (17): 7980-7984; and Kao et al. (2006), Theor. Appl. Genet. 112 (5): 891-902. Sequences and PCR conditions of SSR loci in rice as well as the most current genetic map may be found in RiceBLAST and the TIGR Rice Genome Annotation on the world wide web.

One use of molecular markers is Quantitative Trait Loci (QTL) mapping. QTL mapping is the use of markers which are known to be closely linked to alleles that have measurable effects on a quantitative trait. Selection in the breeding process is based upon the accumulation of markers linked to the positive effecting alleles and/or the elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select for the genome of the recurrent parent and against the genome of the donor parent. Using this procedure can minimize the amount of genome from the donor parent that remains in the selected plants. It can also be used to reduce the number of crosses back to the recurrent parent needed in a backcrossing program. The use of molecular markers in the selection process is often called genetic marker enhanced selection. Molecular markers may also be used to identify and exclude certain sources of germplasm as parental varieties or ancestors of a plant by providing a means of tracking genetic profiles through crosses.

### Double Haploids

The production of double haploids can also be used for the development of plants with a homozygous phenotype in a breeding program involving DG563PVL. For example, a rice plant for which inbred rice line DG563PVL is a parent can be used to produce double haploid plants. Double haploids are produced by the doubling of a set of chromosomes (1 N) from a heterozygous plant to produce a completely homozygous individual. See, e.g., Wan et al. (1989), Theoretical and Applied Genetics, 77:889-892; and U.S. Pat. No. 7,135,615.

### Tissue Culture

Reproduction of inbred rice line DG563PVL can occur by tissue culture and regeneration. Methods of tissue culturing cells of DG563PVL and a tissue culture of DG563 are provided. Tissue culture of various tissues of rice and regeneration of plants therefrom is well known in the art, with methods having been widely published. See, e.g., Komatsuda et al. (1991), Crop Sci., 31:333-337; Stephens et al. (1991), Theor. Appl. Genet., 82:633-635; Shetty et al. (1992), Plant Science, 81:245-251; U.S. Pat. Nos. 5,008,200; and 5,024,944. The present disclosure thus provides regenerative cells which, upon growth and differentiation, produce rice plants having the genotype and/or phenotypic characteristics of inbred rice line DG563PVL.

The term “tissue culture” refers to a composition including isolated cells of the same type or cells of different types, or a collection of such cells organized into parts of a plant. Examples of tissue cultures include protoplasts, calli, plant clumps, and plant cells that can generate tissue cultures that form intact plants or parts of plants, such as embryos, pollen, flowers, seeds, glumes, panicles, leaves, stems, roots, root tips, anthers, and the like. Means for preparing and maintaining plant tissue cultures are well known in the art.

As used herein phrases such as “growing the seed” or “grown from the seed” include embryo rescue, isolation of cells from seed for another use in tissue culture, as well as traditional growing methods.

Introduction of a New Trait or Locus into Inbred Rice Line DG563PVL

Inbred rice line DG563PVL provides a new genetic line into which a new locus, loci, or trait(s) may be introgressed. Direct transformation and backcrossing are two important methods that can be used to accomplish such an introgression.

### Backcross Conversions of Inbred Rice Line DG563PVL

A backcross conversion of inbred rice line DG563PVL occurs when DNA sequences are introduced through backcrossing, with inbred rice line DG563PVL utilized as the recurrent parent. Backcrossing involves the repeated crossing of a DG563PVL hybrid progeny back to one of the parental rice plants (i.e., the recurrent parent). The recurrent parent can be DG563PVL, and backcrossing to the recurrent parent can occur 1, 2, 3, 4, 5, 6, 7, 8, 9 or more times.

Both naturally occurring and transgenic DNA sequences can be introduced through backcrossing techniques. In a typical backcross protocol, the original cultivar of interest (recurrent parent) is crossed to a second cultivar (nonrecurrent parent) that carries the single or multiple gene(s) of interest to be transferred. The resulting progeny from this cross are then crossed again to the recurrent parent and the process is repeated until a rice plant is obtained wherein essentially all of the desired morphological and physiological characteristics of the recurrent parent are recovered in the converted plant, in addition to the single or multiple transferred gene(s) from the nonrecurrent parent.

The selection of a suitable recurrent parent (e.g., DG563PVL) is an important step for a successful backcrossing procedure. The goal of a backcross protocol is to alter or substitute a single or multiple trait or characteristic in the original line. To accomplish this, a single gene or multiple genes of the recurrent line is/are modified or substituted with the desired gene(s) from the nonrecurrent parent, while retaining essentially all of the rest of the desired genetic (and therefore the desired physiological and morphological) constitution of the recurrent line. The choice of the particular non-recurrent parent will depend on the purpose of the backcross; one of the major purposes is to add some commercially desirable, agronomically important trait to the plant. The exact backcrossing protocol will depend on the characteristic(s) or trait(s) being altered to determine an appropriate testing protocol. Although backcrossing methods are simplified when the characteristic(s) being transferred are the result of the action of a dominant allele(s), a recessive allele may also be transferred. In this instance it may be necessary to introduce a test of the progeny to determine if the desired characteristic(s) has been successfully transferred.

Many single or multiple gene traits have been identified that are not regularly selected for in the development of a new line but that can be improved by backcrossing techniques. Single or multiple gene traits may or may not be transgenic. Examples of these traits include but are not limited to, male sterility, waxy starch, herbicide resistance, resistance for bacterial, fungal, or viral disease, insect resistance, male fertility, enhanced nutritional quality, industrial usage, yield stability, and yield enhancement.

A backcross conversion with DG563PVL may produce a plant with a desired trait(s), locus or loci conversion in at least two or more backcrosses, including at least 2 crosses, at least 3 crosses, at least 4 crosses, at least 5 crosses and the like. Molecular marker assisted breeding or selection may be utilized to reduce the number of backcrosses necessary to achieve the backcross conversion. Using marker assisted selection, a backcross conversion can be made in as few as two backcrosses.

The complexity of the backcross conversion method depends on the type of trait being transferred (e.g., single genes or closely linked genes vs. unlinked genes), the level of expression of the trait, the type of inheritance (e.g., cytoplasmic or nuclear) and the types of parents included in the cross. It will be understood by those of ordinary skill in the art that for single gene traits that are relatively easy to classify, the backcross method is effective and relatively easy to manage. Desired traits that may be transferred through backcross conversion include, but are not limited to, sterility (nuclear and cytoplasmic), fertility restoration, nutritional enhancements, drought tolerance, nitrogen utilization, altered fatty acid profile, low phytate, industrial enhancements, disease resistance (bacterial, fungal or viral), insect resistance, and herbicide resistance. In addition, an introgression site itself, such as an FRT site, Lox site or other site-specific integration site, may be inserted by backcrossing and utilized for direct insertion of one or more genes of interest into a specific plant variety. In some embodiments, the number of loci that may be backcrossed into inbred rice line DG563PVL is 1, 2, 3, 4, 5, 6, 7, 8, 9, or 10. A single locus may contain several transgenes, such as a transgene for disease resistance that, in the same expression vector, also contains a transgene for herbicide resistance. The gene for herbicide resistance may be used as a selectable marker and/or as a phenotypic trait. A single locus conversion of a site-specific integration system allows for the integration of multiple genes at the converted loci.

A genetic trait which has been engineered into a particular rice hybrid or cultivar using molecular biology techniques can be moved into another hybrid or cultivar using traditional backcrossing techniques. For example, a backcrossing approach can be used to move an engineered trait from a public, non-elite rice plant into an elite rice plant (e.g., DG563PVL), or from a rice plant containing a foreign gene in its genome into a rice plant which does not contain that gene.

### Foreign Proteins Genes and Agronomic Genes

Techniques for the selection and propagation of transformed rice plants, which are well understood in the art, yield a plurality of transgenic plants which are harvested in a conventional manner. For the relatively small number of transgenic plants that show higher levels of expression, a genetic map can be generated, primarily via conventional RFLP, PCR and SSR analysis, which identifies the approximate chromosomal location of the integrated DNA molecule.

Map information concerning chromosomal location is useful for proprietary protection of a subject transgenic plant. If unauthorized propagation is undertaken and crosses made with other germplasm, the map of the integration region can be compared to similar maps for suspect plants to determine if the suspect plants have a common parentage with the subject plant. Map comparisons can involve hybridizations, RFLP, PCR, SSR and sequencing, each of which are conventional and well known in the art.

Through the transformation of rice, the expression of genes can be altered to enhance, confer, or otherwise alter herbicide resistance; bacterial disease resistance; viral disease resistance; fungal disease resistance; insect resistance; modified fatty acid metabolism; modified carbohydrate metabolism; and other triates. Transformation can also be used to insert DNA sequences which control or help control male-sterility. DNA sequences native to rice as well as non-native DNA sequences can be transformed into rice and used to alter levels of native or non-native proteins. Various promoters, targeting sequences, enhancing sequences, and other DNA sequences can be inserted into the genome for the purpose of altering the expression of proteins. Reduction of the activity of specific genes (also known as gene silencing, or gene suppression) is desirable for several aspects of genetic engineering in plants.

Many techniques for gene silencing are well known to one of skill in the art, including but not limited to knock-outs. Knock-outs can be generated by, for example, insertion of a transposable element such as mu or other genetic elements such as a FRT, Lox or other site specific integration site; antisense technology; co-suppression; RNA interference; virus-induced gene silencing; target-RNA-specific ribozymes; hairpin structures; MicroRNA; ribozymes; oligonucleotide-mediated targeted; zinc-finger nuclease-based gene editing; CRISPR/Cas-based gene editing; TALEN-based gene editing; EMN-base gene editing; and other methods or combinations of the above methods known to those of skill in the art.

Agronomic genes can be expressed in transformed plants. More particularly, plants can be genetically engineered to express various phenotypes of agronomic interest. Exemplary genes implicated in this regard include, but are not limited to, those described below.

### Transformation Methods

Numerous methods for plant transformation have been developed, including biological and physical plant transformation protocols. See, e.g., Miki et al., “Procedures for Introducing Foreign DNA into Plants” in Methods in Plant Molecular Biology and Biotechnology, Glick and Thompson, Eds. (CRC Press, Inc., Boca Raton, 1993), pp. 67-88. In addition, expression vectors and in vitro culture methods for plant cell or tissue transformation and regeneration of plants are available. See, e.g., Gruber et al., “Vectors for Plant Transformation” in Methods in Plant Molecular Biology and Biotechnology, Glick and Thompson, Eds. (CRC Press, Inc., Boca Raton, 1993), pp. 89-119. Plant transformation methods include the natural transformation system of Agrobacterium, or direct gene transfer through microprojectile-mediated transformation or sonication.

Following transformation of target tissues or cells of DG563PVL, expression of at least one gene transferred to the DG563PVL cells can provide for preferential selection of transformed cells, tissues and/or plants, using regeneration and selection methods now well known in the art.

### Gene Editing

Targeted gene editing resulting in gene knock-out, knock-in, down regulation, or altered function can be conducted on inbred rice line DG563PVL or a parent plant to be crossed with DG563PVL. The genome of a rice plant can be edited to modify traits and confer or alter resistances or tolerances to pests, viruses, and herbicides. Several gene editing methodologies are well known in the art, including the clustered regularly interspaced short palindromic repeat (CRISPR)/CRISPR-associate protein system, the transcription activator-like effector nuclease (TALEN) system, the zinc-finger nuclease (ZFN) system, and the engineered homing endonuclease/meganuclease (EMN) system.

### Genetic Marker Mapping

In addition to phenotypic observations, a plant can also be identified by its genotype. The genotype of a plant can be characterized through a genetic marker profile which can identify plants of the same variety or a related variety, or be used to determine or validate a pedigree. Genetic marker profiles can be obtained by techniques known in the art, such as isozyme electrophoresis, restriction fragment length polymorphisms (RFLPs), randomly amplified polymorphic DNAs (RAPDs), arbitrarily primed polymerase chain reaction (AP-PCR), DNA amplification fingerprinting (DAF), sequence characterized amplified regions (SCARs), amplified fragment length polymorphisms (AFLPs), simple sequence repeats (SSRs) (which are also referred to as Microsatellites), and single nucleotide polymorphisms (SNPs).

Particular markers used for these purposes are not limited to any particular set of markers, but are envisioned to include any type of marker and marker profile which provides a means of distinguishing varieties.

Primers and PCR protocols for assaying these and other markers are widely known in the art. In addition to being used for identification of inbred rice line DG563PVL and plant parts and plant cells of inbred rice line DG563PVL, the genetic profile may be used to identify a rice plant produced through the use of inbred rice line DG563PVL or to verify a pedigree for progeny plants produced through the use of inbred rice line DG563PVL The genetic marker profile is also useful in breeding and developing backcross conversions.

An inbred rice line plant characterized by molecular and physiological data obtained from DG563PVL is contemplated.

Means of performing genetic marker profiles using SSR polymorphisms are well known in the art. SSRs are genetic markers based on polymorphisms in repeated nucleotide sequences, such as microsatellites. A marker system based on SSRs can be highly informative in linkage analysis relative to other marker systems in that multiple alleles may be present. Another advantage of this type of marker is that, through use of flanking primers, detection of SSRs can be achieved, for example, by the polymerase chain reaction (PCR), thereby eliminating the need for labor-intensive Southern hybridization. The PCR detection is done by use of two oligonucleotide primers flanking the polymorphic segment of repetitive DNA. Repeated cycles of heat denaturation of the DNA followed by annealing of the primers to their complementary sequences at low temperatures, and extension of the annealed primers with DNA polymerase, comprise the major part of the methodology.

Following amplification, markers can be scored by electrophoresis of the amplification products. Scoring of marker genotype is based on the size of the amplified fragment, which may be measured by the number of base pairs of the fragment. While variation in the primer used or in laboratory procedures can affect the reported fragment size, relative values should remain constant regardless of the specific primer or laboratory used. When comparing hybrids or varieties it is preferable if all SSR profiles are performed in the same lab.

Plants and plant parts substantially benefiting from the use of inbred rice line DG563PVL in their development, such as inbred rice line DG563PVL comprising a backcross conversion, transgene, or genetic sterility factor, may be identified by having a molecular marker profile with a high percent identity to inbred rice line DG563PVL. Such a percent identity might be 95%, 96%, 97%, 98%, 99%, 99.5% or 99.9% identical to inbred rice line DG563PVL.

The SSR profile of inbred rice line DG563PVL also can be used to identify essentially derived varieties and other progeny varieties developed from the use of inbred rice line DG563PVL, as well as cells and other plant parts thereof. Progeny plants and plant parts produced using inbred rice line DG563PVL can be identified by having a molecular marker profile of at least 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.5% identical to DG563PVL. Such progeny may be further characterized as being within a pedigree distance of inbred rice line DG563PVL, such as within 1, 2, 3, 4, or 5 or fewer cross-pollinations to a rice plant other than inbred rice line DG563PVL or a plant that has inbred rice line DG563PVL as a progenitor. Unique molecular profiles may be identified with other molecular tools such as SNPs and RFLPs.

While determining the SSR genetic marker profile of the plants described, several unique SSR profiles may also be identified which did not appear in either parent of such rice plant. Such unique SSR profiles may arise during the breeding process from recombination or mutation. A combination of several unique alleles provides a means of identifying a plant variety, an F1 progeny produced from such variety, and progeny produced from such rice plan.

Thus, a method for preparing a descendent seed comprises planting a seed capable of producing a plant hereof, growing the resulting plant, and harvesting descendent seed thereof. In some embodiments, such a method can further comprise applying an ACCase-inhibiting herbicide composition to the resulting plant.

### Seed Treatments and Cleaning

Methods of harvesting the seed of the inbred rice variety DG563PVL as seed for planting are provided. Embodiments include cleaning the seed, treating the seed, and/or conditioning the seed. Cleaning the seed is understood in the art and include removal of foreign debris such as one or more of weed seed, chaff, and plant matter from the seed. Conditioning the seed is understood in the art to include controlling the temperature and rate of dry down of the seed and storing seed in a controlled temperature environment. Seed treatment is the application of a composition to the seed such as a coating or powder.

Methods for producing treated seed include the step of applying a composition to the seed or seed surface. Seeds are provided which have on the surface a composition. Biological active components such as microorganisms can also be used as a seed treatment. Some examples of compositions are insecticides, fungicides, pesticides, anti-microbials, germination inhibitors, germination promoters, cytokines, and nutrients.

### ACCase-Inhibiting Herbicides

Inbred rice variety DG563PVL is tolerant of herbicides that interfere with acetyl-Coenzyme A carboxylase activity. Specifically, inbred rice variety DG563PVL is tolerant of herbicides containing quizalofop and esters thereof as the active ingredient (A.I.). More specifically, inbred rice variety DG563PVL has shown tolerance to herbicides containing the active ingredient quizalofop-P-ethyl. Commercially available herbicides containing quizalofop-P-ethyl as one of the A.I. include, but are not limited to, Provisia® herbicide (BASF).

Methods of the present disclosure relate to methods for growing a rice plant comprising, providing the seed of the inbred rice variety DG563PVL, applying a weed-killing rate of ACCase-inhibiting herbicide composition to a vicinity around the seed, post-emergencce, thereby creating a treated DG563PVL rice plant; and growing the treated DG563PVL rice plant.

Methods of the present disclosure also relate to methods for controlling weeds in the vicinity of a plant of the inbred rice variety DG563PVL, comprising planting a plurality of seed of the inbred rice variety DG563PVL, applying a weed-killing rate of ACCase-inhibiting herbicide composition to a vicinity around the planted seed, post-emergencce, thereby creating a treated DG563PVL rice plant, and growing the treated DG563PVL rice plant.

In other embodiments the the ACCase-inhibiting herbicide composition is quizalofop or ester thereof. In further embodiments the effective amount of quizalofop or ester thereof is at least 14 g Al/Ha (grams of active ingredient per hectar). In other further embodiments the effective amount of quizalofop or ester thereof is at least 16, 18, 20, 22, 24 or 26 g Al/Ha. In other further embodiments the effective amount of quizalofop or ester thereof is at least 28, 32, 34, 36, 38, or 40 g Al/Ha. In other further embodiments the effective amount of quizalofop or ester thereof is at least 40, 44, 50, 56, 62, 66, or 72 g Al/Ha. In other further embodiments the effective amount of quizalofop or ester thereof is at least 76, 82, 88, 94 or 102 g Al/Ha. In other further embodiments the effective amount of quizalofop or ester thereof is at least 120, 130 or 140 g Al/Ha.

In some embodiments herbicide composition may include the more than one ACCase-inhibiting active ingredient. In further embodiments the herbicide composition may comprise one ACCase-inhibiting active ingredient and one or more different agronomic active ingredients.

In some embodiment, providing a plant or seed of inbred rice variety DG563PVL relates to planting a seed of inbred rice variety DG563PVL and allowing the plant to emerge prior to applying an effective amount of herbicide. In other embodiments, providing a plant of inbred rice variety DG563PVL relates to transplanting the plant of DG563PVL prior to applying an effective amount of ACCase-inhibiting herbicide composition. In still other embodiments, providing a plant of inbred rice variety DG563PVL relates to the DG563PVL plant being established pre-emergence or post-emergence in the field prior to applying an effective amount of herbicide post-emergence.

In some embodiments, the herbicide is applied 2-4 days post-emergence. In some embodiments, the herbicide is applied at the 2 to 3-leaf stage. In some further embodiments, herbicide is applied in one to two applications.

In some embodiments, the application mode of the ACCase-inhibiting herbicide composition is foliar. Those of skill in the art will appreicte that foliar application may be performed via ground or air spray equipment.

### Industrial Uses

In various embodiments, plants hereof can be used to produce plant products. In some embodiments, a method for producing a derived product from a plant hereof can comprising processing a plant part thereof to obtain a derived product. The seed of inbred rice line DG563PVL, the plant produced from the seed, the hybrid rice plant produced from the crossing of the variety with any other rice plant, hybrid seed, and various parts of the hybrid rice plant can be utilized for human food, livestock feed, and as a raw material in industry.

In some embodiments, such a method can be used to obtain a derived product that is any of, e.g., fodder, feed, seed meal, oil, or seed-treatment-coated seeds. Seeds, treated seeds, and other plant products obtained by such methods are useful products that can be commercialized. The rice seeds produced by inbred rice line DG563PVL can be crushed, or a component of the rice seeds can be extracted, in order to comprise a commodity plant product, such as protein concentrate, protein isolate, meal, flour, starch, extract, or oil for a food or feed product.

Inbred rice line DG563PVL can be used as a food source for both animals and humans. Rice and rice by-products (rice bran, cracked rice) are used as a food for livestock, including cattle and other ruminants.

Inbred rice line DG563PVL can be used to produce rice meal. Following milling of rice harvested from inbred rice line DG563PVL, the remaining material or “meal” is collected. The meal includes rice bran, polishings, and some rice flour. Rice meal is primarily used as a feedstuff for livestock.

Inbred rice line DG563PVL can be used to produce rice flour.

For human consumption, inbred rice line DG563PVL can be used to produce cooking rice, or other product such as rice flour (used to make, e.g., noodles, rice paper, cake, and dumplings), alcoholic beverages, rice vinegar, rice milk, rice syrup, puffed rice, rice crackers, and rice starch.

Inbred rice line DG563PVL can further be processed to produce films, packaging, and nutraceutical products.

In various embodiments, the present disclosure provides production of food products, consumer products, industrial products, and veterinary products from any of the plants described herein.

### Deposit Information

Applicant has made a deposit of the required number of seeds of Inbred Rice Line DG563PVL with the National Laboratory for Genetic Resources Preservation (NLGRP), Fort Collins, CO, USA, Deposit No. PI 706255. The seeds deposited with the NLGRP on Nov. 12, 2024 were taken from the deposit maintained by Loveland Products, Inc. at LPI Rice Breeding Station in El Campo, TX since prior to the filing date of this application. Access to this deposit will be available during the pendency of the application to the Commissioner of Patents and Trademarks and persons determined by the Commissioner to be entitled thereto upon request. Upon allowance of any claims in the application, the Applicant will make the deposit available to the public pursuant to 37 CFR § 1.808. This deposit of the Inbred Rice Line DG563PVL will be maintained in the NLGRP depository, which is a public depository, for a period of 30 years, or 5 years after the most recent request, or for the enforceable life of the patent, whichever is longer, and will be replaced if it becomes nonviable during that period. Additionally, Applicant has or will satisfy all the requirements of 37 CFR §§ 1.801-1.809, including providing an indication of the viability of the sample upon deposit. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

What is claimed is:

1. A seed, plant, plant part, or plant cell of inbred rice variety DG563PVL, representative seed of the variety having been deposited with National Laboratory for Genetic Resources Preservation (NLGRP) under deposit number PI 706255.

2. A rice plant or a plant part thereof, produced by growing the seed of the inbred rice variety DG563PVL of claim 1.

3. An F1 rice seed of rice variety DG563PVL produced by crossing the plant or plant part of claim 1 with a plant of a different rice variety.

4. A method of producing a rice seed derived from the variety DG563PVL, comprising,
a) crossing the plant of claim 1 with itself or a second plant to produce progeny seed; and
b) growing the progeny seed to produce a progeny plant and crossing the progeny plant with itself or a different plant to produce rice seed derived from the variety DG563PVL.

6. The method of claim 4 further comprising selecting the progeny plants by applying a weed-killing rate of ACCase-inhibiting herbicide during the growing of the progeny seed.

7. A method for growing a rice plant comprising,
a) providing the seed of the inbred rice variety DG563PVL of claim 1;
b) applying a weed-killing rate of ACCase-inhibiting herbicide composition to a vicinity around the seed, post-emergencce, thereby creating a treated DG563PVL rice plant; and
c) growing the treated DG563PVL rice plant.

8. The method of claim 7 wherein the ACCase-inhibiting herbicide composition is quizalofop-P-ethyl.

9. The method of claim 8 wherein the ACCase-inhibiting herbicide composition is applied at a rate of in the range of 14-140 g Al/ha.

10. The method of claim 8 wherein the application of the ACCase-inhibiting herbicide composition is foliar.

11. The method of claim 8 wherein the ACCase-inhibiting herbicide composition is applied 2-4 days post-emergence.

12. A method for controlling weeds in the vicinity of a plant,
a) planting a plurality of seed of the inbred rice variety DG563PVL of claim 1;
b) applying a weed-killing rate of ACCase-inhibiting herbicide composition to a vicinity around the planted seed, post-emergencce, thereby creating a treated DG563PVL rice plant; and
c) growing the treated DG563PVL rice plant.

13. The method of claim 12 wherein the ACCase-inhibiting herbicide composition is quizalofop-P-ethyl.

14. The method of claim 13 wherein the ACCase-inhibiting herbicide composition is applied at a rate of in the range of 14-140 g Al/ha.

15. The method of claim 13 wherein the application of the ACCase-inhibiting herbicide composition is foliar.

16. The method of claim 13 wherein the ACCase-inhibiting herbicide composition is applied 2-4 days post-emergence.

17. A method of making a commodity plant product comprising rice, rice meal, rice flour, or rice oil, the method comprising producing the commodity pant product from the rice plant or plant part of claim 1.

18. A commodity plant product derived or produced from the rice plant of claim 2.

19. A commodity plant product derived or produced from the rice plant of claim 7.

20. A commodity plant product derived or produced from the rice plant of claim 12.

21. A commodity plant product of claim 17, wherein the commodity plant product comprises at least one cell of a rice plant of inbred rice line DG563PVL.

22. A commodity plant product of claim 17, wherein the commodity plant product is rice, rice meal, rice flour, or rice oil.
